(19) United States
(12) Patent Application Publication
(10) Pub. No.: US 2017/0009257 A1
(43) Pub. Date: Jan. 12, 2017

(54) BIOMASS TRANSFORMATION

(71) Applicant: DEINOVE, Grabels (FR)

(72) Inventors: FABIEN COZE, MONTPELLIER (FR); NAGIB WARD, WAKE FOREST, NC (US); ASSIA DREUXZIGHA, MONTPELLIER (FR); CLARINE LAZZARELLI, JUVIGNAC (FR); JEAN-PAUL LEONETTI, CASTELNAU LE LEZ (FR)

(21) Appl. No.: 15/117,742

(22) PCT Filed: Feb. 13, 2015
(86) PCT No.: PCT/EP2015/053158

§ 371 Date: Aug. 10, 2016

(30) Foreign Application Priority Data
Feb. 14, 2014 (EP) 14305204.1

(51) Int. Cl. C12P 1/04; C12P 7/04

(52) U.S. Cl. CPC C12P 1/04; C12P 7/04; C12P 2201/00

(57) ABSTRACT

The present invention provides improved methods of transforming biomass using Deinococcus bacteria. More particularly, the invention discloses improved methods that combine suitable biomass treatment and processing conditions, allowing transformation of biomass and generation of valuable products under industrially-effective conditions

The present invention provides improved methods of transforming biomass using Deinococcus bacteria. More particularly, the invention discloses improved methods that combine suitable biomass treatment and processing conditions, allowing transformation of biomass and generation of valuable products under industrially-effective conditions.

Introduction

Biomasses such as lignocellulosic biomass represent a very abundant substrate from which valuable products may potentially be derived, including e.g., fermentable sugars, metabolites, proteins, biofuels, etc., with applications in several fields such as chemical, pharmaceutical, biotechnological, and food industries. Transforming biomasses into such products under industrially suitable conditions is, however, a tremendous challenge. In particular, biomasses are diverse and complex materials, composed of different polymers of various degrees of polymerization and complexity, which are not easily transformed by microorganisms. In addition, biomasses are high volume materials, which require very large scale treatment conditions and installations to ensure proper transformation, which are generally not adapted to microorganism. Moreover, biomasses may need to be processed under stringent conditions (temperature, pH, etc.) that could be incompatible with microorganism culture conditions.

Various methods have been discussed in the art to modify biomass in order to improve their cellulose content and/or to derive products therefrom. Such methods involve chemical and/or physical pre-treatments, enzymatic digestion, and/or biological degradation. Such methods may be reviewed, for instance, in Alvira et al (Bioresource Technology 101 (2010) 4851-4861). WO2011/028554 discloses a method comprising removing from the biomass fine particles with a screen having openings of a size of about 840 μm to produce a cleaned biomass feedstock having an ash content of no more than about 75%. Furthermore, methods have been reported in the art which combine chemical treatment with an enzymatic or microbial digestion. In this regard, many different types of microorganisms have been grown or engineered in vitro for such uses, including a vast number of bacteria (e.g., E. coli, Bacillus, Rhodobacter, Zymomonas, Deinococcus, etc.), algae, fungi (Trichoderma, Aspergillus, Fusarium) or yeasts (Saccharomyces, Kluyveromyces, etc.). However, there remain major limitations with the existing methods. In particular, while a number of microorganisms may exhibit fermentative activity in vitro under laboratory conditions, there has been no demonstration in the art of a potent fully-integrated process for effective transformation of biomass under industrially applicable conditions. In particular, effective transformation of biomass requires treatment of very large amounts of substrate at effective costs and under fermentative conditions. The large volumes of materials to be treated require stringent maintenance conditions (acidity, temperature) in order to avoid contamination. In addition, under such industrial conditions, large amounts of solvents, xenobiotics, or growth inhibitors may be released during fermentation, which shall alter the effectiveness of the process. Moreover, the transformation of biomass requires several steps including altering the integrity of biomass constituents (such as polymers of lignin, cellulose, etc.), their hydrolysis into sugars and the fermentation of the sugars. All of these steps may require specific treatment conditions and reagents which could be incompatible with each other. In particular, cellulase enzymes are known to be inhibited by products such as glucose or cellobiose. Similarly, furfural, generated by the biomass, is toxic to microorganisms. Also, most microorganisms grow at temperatures where enzymes are not optimally active.

Accordingly, despite substantial progress and huge interest in the field, there is still a need for optimized fully integrated and operational methods of transforming biomass.

### SUMMARY OF THE INVENTION

The present invention provides improved methods of transforming biomass using Deinococcus bacteria. More particularly, the invention discloses improved methods that combine suitable biomass treatment and processing conditions, allowing transformation of biomass and generation of valuable products under industrially-effective conditions.

It is therefore an object of the invention to provide a method of transforming a biomass, comprising:

a) subjecting the biomass to a treatment to increase the hydrolytic power thereof (e.g., increase the amount or accessibility or solubility or digestibility of cellulose and/or hemicellulose and/or starch; and/or alter crystalline structure of cellulose);

b) simultaneously with or subsequently to step a), exposing the biomass to an enzymatic activity that degrades polysaccharides in the biomass, preferably at least a cellulase and/or a hemicellulase and/or an oxidoreductase and/or an amylase and/or an esterase and/or a pectinase; and

c) simultaneously with or subsequently to step b), exposing the biomass to a Deinococcus bacterium or an extract thereof. Said exposition allows fermentation of substrates in the biomass, leading to effective transformation of the biomass.

Steps a)-c) may be carried out sequentially or in combination(s). In a particular embodiment, steps a), b) and c) are conducted simultaneously. In another, preferred, embodiment, steps b) and c) are performed simultaneously, after step a). In a further particular embodiment, steps a), b) and c) are conducted sequentially, in a same or distinct reactor.

Preferably, steps a) to c) are performed in a liquid medium, and may optionally further include a solid/liquid separation.

The pH in steps a) to c) may be comprised between 3 and 9, preferably between 4 and 6.5, even more preferably between 4.5 and 6, most preferably between 5-6.

Steps b) and c) may be performed at a temperature comprised between 40 and 65° C., preferably 45 and 60° C., more preferably 48-55° C., even more preferably about 50° C.

A further object of the invention relates to a method of transforming a biomass, comprising:

a) providing a pre-treated biomass (i.e., having hydrolytic power);

b) exposing the pre-treated biomass to an enzymatic activity that degrades polysaccharides, preferably at least a cellulase and/or a hemicellulase and/or an oxidoreductase and/or an esterase and/or a pectinase; and

c) simultaneously with or subsequently to step b), exposing the biomass to a Deinococcus bacterium or an extract thereof. Said exposition allows fermentation of substrates in the biomass, leading to effective transformation of the biomass.

As will be disclosed, biomass pre-treatment may include any chemical and/or physical pretreatment such as, without limitation, heat and/or pressure treatment, steam explosion, organosolv treatment, pressurized hot water, supercritical water, ammonia fiber explosion, mild acid, strong acid, mild alkali treatment (less than 4% (w/w) of base, e.g. 0.5-4% (w/w)) or strong alkali treatment (4-20% of base (w/w), preferably 6-20%, i.e. preferably at a pH comprised between 10 and 13), gas injection such as ozone (ozonolysis), CO₂ explosion, or combinations thereof. Preferably, the treatment includes mild acid, strong acid, mild alkali, strong alkali treatment or ammonia fiber explosion, or combinations thereof. More preferably, the treatment includes mild acid, strong acid, mild alkali or strong alkali treatment. Most preferred treatment includes an acid treatment at a pH comprised between 3 and 6, preferably comprised between 3 and 5, preferably during 1 to 24 hours and/or a transient thermic (e.g. from 80 to 150° C.) and/or pressure (e.g. from 40 to 200 psigs) treatment.

A further object of the invention relates to a method for transforming a biomass comprising providing a biomass and exposing the biomass to a Deinococcus bacterium in an acid medium in the presence of one or several enzymes that degrade cellulose and/or hemicellulose.

A further object of the invention relates to a method for transforming a biomass comprising providing the biomass, heating the biomass, optionally under pressure, and exposing the biomass to a Deinococcus bacterium in the presence of one or several amylases.

A further object of the invention relates to a method of producing an alcohol, comprising:

a) subjecting a biomass to a treatment to increase the hydrolytic power thereof;

b) simultaneously with or subsequently to step a), exposing the biomass to an enzymatic activity that degrades polysaccharides in the biomass;

c) simultaneously with or subsequently to step b), exposing the biomass to a Deinococcus bacterium or an extract thereof; and

d) collecting the alcohol produced.

A further object of the invention relates to a method of producing an alcohol, comprising:

a) providing a pre-treated biomass having hydrolytic power;

b) exposing the pre-treated biomass to an enzymatic activity that degrades polysaccharides in the biomass;

c) simultaneously with or subsequently to step b), exposing the biomass to a Deinococcus bacterium or an extract thereof, and

d) collecting the alcohol produced.

A further object of the invention relates to a method of producing an isoprenoid compound, comprising:

a) subjecting a biomass to a treatment to increase the hydrolytic power thereof;

b) simultaneously with or subsequently to step a), exposing the biomass to an enzymatic activity that degrades polysaccharides in the biomass;

c) simultaneously with or subsequently to step b), exposing the biomass to a Deinococcus bacterium or an extract thereof; and

d) collecting an isoprenoid compound produced.

A further object of the invention relates to a method of producing an isoprenoid compound, comprising:

a) providing a pre-treated biomass having hydrolytic power;

b) exposing the pre-treated biomass to an enzymatic activity that degrades polysaccharides in the biomass;

c) simultaneously with or subsequently to step b), exposing the biomass to a Deinococcus bacterium or an extract thereof, and

d) collecting an isoprenoid compound produced.

A further object of the invention relates to a method of producing an organic acid, comprising:

a) subjecting a biomass to a treatment to increase the hydrolytic power thereof;

b) simultaneously with or subsequently to step a), exposing the biomass to an enzymatic activity that degrades polysaccharides in the biomass;

c) simultaneously with or subsequently to step b), exposing the biomass to a Deinococcus bacterium or an extract thereof; and

d) collecting an organic acid produced.

A further object of the invention relates to a method of producing an organic acid, comprising:

a) providing a pre-treated biomass having hydrolytic power;

b) exposing the pre-treated biomass to an enzymatic activity that degrades polysaccharides in the biomass;

c) simultaneously with or subsequently to step b), exposing the biomass to a Deinococcus bacterium or an extract thereof, and

d) collecting an organic acid produced.

The invention may be used to transform various biomasses and is particularly suited to transform any biomass that comprises cellulose, hemicellulose, lignin, xylan, starch or pectin. Preferred biomasses include lignocellulosic vegetal material or starch-containing organic material, in particular municipal solid wastes (MSW) and agricultural or forestry residues or fractions thereof.

### DETAILED DESCRIPTION OF THE INVENTION

As indicated, the present invention relates to novel methods of transforming biomass using Deinococcus bacteria, particularly to fully integrated optimized industrial processes of transforming biomass using Deinococcus bacteria.

Deinococcus bacteria have proposed for use in industrial processes or reactions using biomass (see e.g., WO2009/063079; WO2010/094665 or WO2010/081899). These applications show that Deinococcus bacteria can be used to produce valuable products from biomass. They also show that Deinococcus has unique properties of efficient substrate assimilation and solvent resistance. The invention stems, inter alia, from the selection and design of particular process conditions which allow optimized efficacy of biomass transformation, suitable for cost-effective industrial use. The invention also stems from the identification of biological pathways in Deinococcus bacteria, such as assimilation of highly polymerized constituents and a tolerance to xenobiotics, that can allow the design of optimized integrated processes for transforming biomass.

More specifically, the invention relates to a method of transforming a biomass comprising

a) subjecting the biomass to a treatment to increase the hydrolytic power thereof;

b) simultaneously with or subsequently to step a), exposing the biomass to an enzymatic activity that degrades polysaccharides in the biomass; and

c) simultaneously with or subsequently to step b), exposing the biomass to a Deinococcus bacterium or an extract thereof.

The method may comprise a further step of collecting a product resulting from said transformation, such as a protein, lipid, gas, nucleic acid or small molecule. The product may be a biofuel (e.g., ethanol), a biologically-active molecule, a metabolite (e.g., an isoprenoid compound, an organic acid), an intermediate molecule, block polymer, etc. Collection of the product can be continuous or sequential.

Definitions

“Transforming” a biomass designates, within the context of the invention, any modification of the structure and/or composition of a biomass. Transforming therefore includes degrading (at least partially); hydrolyzing, denaturing, recycling, metabolizing, and the like. The term “transforming” also includes a method wherein the biomass is used as a substrate to produce a molecule of interest.

“Reactor” designates any device or installation or facility suitable for maintaining and transforming biomass and/or fermenting sugars. A reactor may comprise inlet and outlet devices to supply/collect medium, nutrients, gas, etc. The reactor may be closed or open, such as a tank.

An “extract of a bacterium” designates any fraction obtained from a bacterium, such as a cell supernatant, cell debris, cell walls, DNA extract, enzymes or enzyme preparation or any preparation derived from bacteria by chemical, physical and/or enzymatic treatment, which is essentially free of living bacteria. Preferred extracts are enzymatically-active extracts.

As used herein, the term “biofuel” designates a fuel derived from a living or recently dead biological carbon source and, in particular from biomass as defined below. The biofuel according to the invention comprises “first generation biofuel” and/or “second generation biofuel”. The main source for the production of first generation biofuels are edible plants or parts thereof whereas the second generation biofuels are produced preferably from non-edible plants or non-edible parts of plants such as non food crops, biomass wastes, stalks of wheat, corn or wood. Preferably, the biofuel is a second generation biofuel. Examples of biofuels include, but are not limited to, vegetable oil, biodiesel, bioalcohols, biogas, syngas, solid biofuels and cellulosic biofuels. A preferred biofuel is a bioalcohol. Examples of bioalcohols include, but are not limited to, ethanol, propanol, butanol, glycerol, butanediol and propanediol. Preferably, the biofuel is ethanol.

The terms “isoprenoid compound”, “isoprenoid”, “terpene compound”, “terpene”,“terpenoid compound”, and “terpenoid” are used interchangeably herein and designate a compound derived from isoprene building block and that is capable of being derived from isopentyl diphosphate (IPP). Isoprenoids include metabolites such as sterol, carotenoids, polyprenol substituents of dolichols, quinones and protein. Examples of isoprenoids include, but are not limited to, hemiterpenes (derived from 1 isoprene unit) such as isoprene; monoterpenes (derived from 2 isoprene units) such as myrcene; sesquiterpenes (derived from 3 isoprene units) such as amorpha-4,11-diene and artemisinin; diterpenes (derived from four isoprene units) such as taxadiene or taxol; triterpenes (derived from 6 isoprene units) such as squalene; tetraterpenes (derived from 8 isoprenoids) such as β-carotene; and polyterpenes (derived from more than 8 isoprene units) such as polyisoprene. Isoprenoids find commercial application for example in pharmaceuticals, nutritional, fragrance, flavoring compounds, agricultural pest control agents or biofuels.

As used herein, the term “organic acid” refers to an organic acid, their salts and esters. Preferred organic acids are acetic acid, propionic acid, pyruvic acid, butyric acid, lactic acid and/or succinic acid.

Biomass

Although the invention can be used with any biomass, it is particularly suited for transforming vegetal biomass, more particularly lignocellulosic biomass, i.e., a biomass which comprises lignin, cellulose, or hemicellulose, or starch-containing biomasses. Examples of lignocellulosic biomasses include, more preferably, forestry products, woody feedstock (softwoods and hardwoods), agricultural wastes and plant residues (such as corn stover, shorghum, sugarcane bagasse, sugarcane molasse, grasses, rice straw, wheat straw, empty fruit bunch from oil palm and date palm, agave bagasse, from tequila industry), perennial grasses (switchgrass, miscanthus, canary grass, erianthus, napier grass, giant reed, and alfalfa), and municipal solid waste (MSW), aquatic products such as algae and seaweed, paper, leather, cotton, hemp, natural rubber products, and food or feed processing by-products.

Preferred biomasses include lignocellulosic vegetal material or starch-containing organic material, in particular municipal solid wastes and agricultural or forestry residues, or fractions thereof such as, for instance, domestic, food, kitchen or urban wastes. Specific examples of preferred biomasses for use in the invention are corn stover, corn cobs, wheat straw, bagasse, softwood, hardwood, citrus peels, MSW or any fraction thereof, particularly domestic, food, kitchen or urban wastes.

Step a)

Step a) of the method comprises treating a biomass to increase its hydrolytic power. The term hydrolytic power designates the amount or accessibility or digestibility or solubility of cellulose and/or hemicellulose and/or starch in the biomass. Such includes, for instance, breaking the lignin barrier and/or altering or disrupting the crystalline structure of cellulose and/or solubilizing starch and/or hemicellulose fibers, for instance. The objective of step a) is mainly to hydrolyze or facilitate subsequent hydrolysis of hemicellulose, cellulose and/or starch by e.g., breaking down the crystallinity of cellulose to turn it into a digestible polymer and to remove or break the lignin barrier. An “increase” in hydrolytic power is, more particularly, an increase by at least 5%, even more preferably at least 7%, 10%, 15%, 20%, 25% or more, as compared to untreated biomass. Such an increase can be measured by the amount of cellulose, hemicelluloses or starch in the biomass, or by the amount of sugars that can be generated from the treated vs non-treated biomass.

Biomass treatment may be performed by various techniques, including chemical and/or physical treatments. More particularly, the treatment can comprise steam, heat, pressure, pressurized hot water, supercritical water, mild acid, strong acid, and/or strong alkaline (e.g., NH3) treatment. Ozone or gases may be used as well. In addition, physical treatments such as drying and/or granulometric separation may be conducted as an initial step, for instance in a BRS screw. The specific conditions of the treatment can be adjusted depending on the type of biomass material.

In a preferred embodiment, treatment step a) includes a thermo-chemical treatment such as steam explosion followed by chemical methods with alkali or acids. Thermo-chemical methods cause disruption of the material's structure, degradation of hemicelluloses and cellulose and lignin transformation, thus facilitating the subsequent hydrolysis of cellulose. Most preferred thermo-chemical methods include:
steam explosion
alkaline pre-treatment
dilute acid hydrolysis
organosolv treatment
ammonia fiber explosion (AFEX)
liquid hot water (LHV)

Steam explosion causes explosion of the material due to high temperature and pressure. Typically, the temperature is ranging from 160 to 200° C. and the pressure ranges from 0.69-4.83 MPa. The treatment is performed for a period of time ranging from 2 to 30 min and some catalyst (alkali or acid) may be present such as H₂SO₄, SO₂. Steam explosion may be used to cause lignin transformation and solubilization of hemicellulose.

Alkaline treatment, such as dilute NaOH treatment, removes acetyl group linkage between lignin and hemicellulose and uronic acid substitutions of hemicelluloses, therefore removing lignin from biomass, and allows destructuration of lignin. It is mainly used to recover (partially degraded) cellulose. Lime (Ca(OH)₂) or KOH may also be used and addition of an oxidant such as H₂O₂ to alkaline pre-treatment may improve the performance by favouring lignin removal. In alkaline pretreatment, lignocellulosic materials may be mixed also with bases including hydroxides of potassium, sodium, and calcium, as well as sodium carbonate (Na₂CO₃), ammonia. Low alkali concentrations (<4% w/w) are mostly used at high temperatures and pressures. Mild alkali pretreatment of biomass favours enzymatic hydrolysis especially for materials that have relatively low lignin content (for review see e.g. Bensah & Mensah, 2013, International Journal of Chemical Engineering). Low NaOH concentration pre-treatment is typically 0.5-4% (w/w) and high NaOH concentration between 6-20% (w/w).

Organic or aqueous-organic solvents as well as catalyst such as oxalic, salysilic, acetylsalicylic acids, may be used in the organosolv treatment of biomass, typically at temperature of 150-200° C. A variety of organic solvents such as esters, ketones, organics acids, phenols, and ethers may be also used.

Acid hydrolysis is the most preferred treatment for use in the present invention. Acid hydrolysis may be conducted with mild (pH 5-6.5) or strong (pH 3-5) acid treatment, using any suitable acid(s) such as sulphuric acid, hydrochloric acid, phosphoric acid or nitric acid. Acid hydrolysis can be performed at high temperature (e.g., 120-200° C., for instance 180° C.) during a short period of time (e.g., from 1-15 min), or at lower temperature (e.g., from 50-90° C.) for longer period of time (e.g., 30-90 min).

In a preferred embodiment, step a) comprises an acid treatment of the biomass, particularly a dilute acid treatment of the biomass, producing an acid impregnated biomass. In a particular embodiment, the biomass is incubated with a 1-10% sulphuric acid solution for a period of time comprised between 15 min and 10 hours, preferably 30 min and 4 hours. Temperature may be comprised between 50 and 180° C., for instance. In a particular embodiment, the treatment comprises a transient thermic treatment, such as a treatment at a temperature comprised between 100-200° C. The acid treatment is preferably conducted under stirring and may be followed by acid neutralization prior to step b).

In a particular embodiment, step a) comprises treating the biomass with an acid under stirring at a temperature comprised between 30 and 200° C., for a period comprised between 1 h and 96 30 hours.

In another particular embodiment, step a) comprises treating the biomass with hot water under pressure (e.g. from 40 to 200 psigs).

In another particular embodiment, step a) comprises heating the biomass at a temperature comprised between approx. 80 and 150° C., more preferably of about 120° C., for a period of time comprised between 15 minutes and 4 hours. Optionally such heating may be combined to a pressure treatment (e.g. from 40 to 200 psigs). Such a treatment is particularly suited to increase the hydrolytic power of starch-containing biomass such as domestic biowastes.

Treatment step a) may further comprise one or more additional initial treatment steps, typically performed prior to the above chemical/physical treatments, to improve the characteristics of the biomass. Such additional initial step(s) may include, for instance:
drying and/or granulometric separation; or
passing through a BRS screw; or
broying; or
filtering to remove fine particles, such as ash.

Preferably, the biomass is washed and/or neutralized after treatment step a).

Step b)

Step b) comprises exposing the biomass to one or more enzymatic activities that degrade polysaccharides in the biomass. The enzymes preferably comprise at least a cellulase and/or a hemicellulase and/or an amylase and/or an oxidoreductase and/or an esterase and/or a pectinase.

Such enzymes can, alone or in combination(s), induce or stimulate the hydrolysis of polysaccharides in the biomass, particularly of cellulose or hemicellulose or starch.

In this regard, in a preferred embodiment, step b) comprises exposing the biomass to at least a cellulase.

Within the context of the invention, a [symbol] cellulase [symbol] designates any enzyme which can contribute to cellulose degradation, particularly of the structure or conformation of cellulose. Examples of cellulases include cellobiohydrolases (or exoglucanase; EC 3.2.1.91), endocellulases (or endoglucanase; EC 3.2.1.4), beta-glucosidases (EC 3.2.1.21), cellobiose dehydrogenase ( [symbol] CDH [symbol] EC 1.1.99.18) or polysaccharide mono-oxygenases. In a particular embodiment, a cellobiohydrolase is used. In another embodiment, an endocellulase is used. In a further embodiment, a beta-glucosidase is used. In a preferred embodiment, a mixture of at least two cellulases is used, for instance a cellobiohydrolase and a beta-glucosidase. In a more particular embodiment, a mixture comprising a cellobiohydrolase, a beta-glucosidase and an endocellulase is used.

In another preferred embodiment, step b) comprises exposing the biomass to at least a hemicellulase.

Within the context of the invention, a [symbol] hemicellulase [symbol] designates any enzyme which can contribute to the degradation of hemicellulose, particularly to an alteration of the structure or conformation of hemicellulose. Examples of hemicellulases include xylanase, endo-xylanase (EC 3.2.1.8), mannanases (EC 3.2.1.78), mannosidases (EC 3.2.1.25), beta-D-xylosidases (EC3.2.1.37), and debranching enzymes which hydrolyze side groups branched on hemicellulose backbone, such as alpha-D-glucuronidases (EC: 3.2.1.139 or EC 3.2.1.131), alpha-L-arabinofuranosidases (EC: 3.2.1.55), acetyl xylan esterases (EC: 3.1.1.72) or feluric acid esterases or feruloyl esterases (EC: 3.1.1.73), alpha-fucosidase (EC3.2.1.51), p-coumaroyl esterase (EC 3.1.1.73) and alpha-galactosidase (EC 3.2.1.22).

In a preferred embodiment, at least a xylanase is used.

In another particular embodiment, a xylanase is used in combination with a debranching enzyme. The use of a debranching enzyme is particularly effective when the biomass contains substituted or ramified hemicellulose polysaccharides. Examples of such biomass include, but are not limited to, corn stover which comprises glucuronoarabinoxylan containing α-D-glucuronic (and 4-O-methyl-α-D-glucuronic) acid and α-L-arabinose, and which can be hydrolyzed using α-D-glucuronidase. Also, a mannanase is effective when the biomass comprises softwood which comprises a mannose backbone. An alpha-galactosidase is effective when the biomass comprises softwood containing, for example, galactoglucomannan. An alpha-arabinofuranosidase is also effective when the biomass comprises wheat straw which comprises arabinoxylan which has only side chains of single terminal units of α-L-arabino furanosyl substituents.

In another preferred embodiment, step b) comprises exposing the biomass to at least a cellulase and a hemicellulase. In a particular embodiment, the biomass is contacted with at least a xylanase and a cellulase.

In a particular embodiment, in addition to the above cellulase(s) and/or hemicellulase(s), the biomass is exposed in step b) to at least a further enzymatic activity selected from an oxido-reductase, an esterase, a pectinase and/or a beta-glucanase (endo-1,3(4)).

In another embodiment, the biomass is exposed to an amylase to hydrolyze starch.

Within the context of the invention, an [symbol] amylase [symbol] designates any enzyme which can contribute to the degradation of starch, particularly of the structure or conformation of starch. Examples of amylase include, alpha-amylase (EC3.2.1.1), beta-amylase (EC 3.2.1.2), and gamma-amylase (EC 3.2.1.3), alpha-glucosidase (EC 3.2.1.20), glucoamylase (EC 3.2.1.3), Pullulanase (or pullulan-6-glucanohydrolase, i.e. debranching enzyme; EC 3.2.1.41), isopullulanase (EC 3.2.1.57) and isoamylase (EC 3.2.1.68).

The enzymes in the culture medium shall be present in an amount sufficient to cause a hydrolysis of polysaccharides in the medium, particularly of cellulose and/or hemicellulose and/or starch. In this regard, preferred amounts of enzymes are comprised between 50 mg/kg dry biomass and 50 g/kg dry biomass. Also, the incubation time shall preferably be comprised between 2 and 96 hours, depending on the nature and amount of enzymatic activity, typically between 24-80 hours.

Exposing the biomass to an enzymatic activity may comprise adding one or several enzymes to the biomass (exogenous supply), and/or inducing or allowing expression of one or several enzymes from a microorganism which is added to the biomass. As used herein, the term “exogenous enzyme” designates any enzyme that is supplied to the biomass and is not produced by a microorganism which is added to the biomass.

In a particular embodiment the biomass is exposed to one or several microorganisms which produce, naturally or recombinantly, one or more enzymatic activities required to degrade polysaccharides. In a most preferred embodiment, such a microorganism is a Deinococcus bacterium, particularly a Deinococcus bacterium of step c).

Accordingly, in a preferred embodiment, step b) utilizes one or several Deinococcus bacterium which either naturally or recombinantly produce enzymes that hydrolyze polysaccharides. In a particular embodiment, a Deinococcus is used which produces a cellulase. In another particular embodiment, a Deinococcus is used which produces a hemicellulase. In this regard, the inventors have shown that Deinococcus sp. have naturally xylanolytic and or cellulolytic activities, allowing to decrease/eliminate the amount of added exogenous enzyme(s). The preparation of such recombinant Deinococcus bacteria can be made as described for instance in WO2013/092965, which is incorporated therein by reference.

Furthermore, in addition to such microorganisms, enzymes by further be added to the biomass, to further increase the enzymatic activity or diversity.

Step c)

Step c) comprises exposing the biomass to one or more Deinococcus bacteria, or extracts thereof. The method of the invention can be performed using various native or modified Deinococcus species, such as, without limitation, D. geothermalis, D. radiodurans, D. cellulolysiticus, D. murrayi, D. guilhemensis, D. aerius, D. aerolatus, D. aerophilus, D. aetherius, D. alpinitundrae, D. altitudinis, D. apachensis, D. aquaticus, D. aquatilis, D. aquiradiocola, D. caeni, D. claudionis, D. daejeonensis, D. depolymerans, D. deserti, D. erythromyxa, D. ficus, D. frigens, D. gobiensis, D. grandis, D. hohokamensis, D. hopiensis, D. humi, D. indicus, D. maricopensis, D. marmoris, D. misasensis, D. mumbaiensis, D. navajonensis, D. papagonensis, D. peraridilitoris, D. pimensis, D. piscis, D. proteolyticus, D. radiodurans, D. radiomollis, D. radiophilus, D. radiopugnans, D. reticulitermitis, D. roseus, D. saxicola, D. sonorensis, D. wulumuqiensis, D. xibeiensis, D. xinjiangensis, D. yavapaiensis and D. yunweiensis.

In a preferred embodiment, the method of the invention uses a thermophilic Deinococcus.

As mentioned above, Deinococcus strains as used in the present application can be used either in native form, or modified (e.g., chemically or genetically) to acquire improved properties. In this regard, in a particular embodiment, the method uses a Deinococcus bacterium that is modified by mutagenesis, by accelerated evolution or by DNA shuffling technologies or by insertion of eucaryote, prokaryote or synthetic non-Deinococcus DNA or by insertion of another Deinococcus strain DNA, said modification affecting viability, growth or functions of the said bacterium in order to promote the modification of biomass or to improve the xenobiotic agent (such as furfural) or organic solvent (such as ethanol) resistance.

Most preferred bacteria for use in the invention are thermophilic Deinococcus bacteria and/or Deinococcus bacteria that can simultaneously utilize C6 and C5 sugars. Examples of C5 sugars include xylose and arabinose and examples of C6 sugars include glucose, cellobiose, galactose and starch. The inventors have discovered that Deinococcus bacteria can simultaneously utilize C6 and C5 sugars, therefore substantially improving the performances of the process. Such Deinococcus bacteria are particularly useful since they can conduct co-fermentation of e.g., glucose and xylose; and/or co-fermentation of glucose and cellobiose; and/or co-fermentation of glucose and/or xylose and/or arabinose, in particular D-xylose and/or L-arabinose.

In addition, the inventors have discovered Deinococcus bacteria that may also ferment small xylo- and/or cellulo-oligosaccharides or cellulodextrines of different degree of polymerization (such as xylobiose, xylotriose, etc.). The use of such bacteria therefore further increases the performance of the process, by allowing more efficient and complete transformation of biomass, while reducing the need for added exogenous enzymes.

In a further preferred embodiment, the invention uses Deinococcus bacteria which are resistant or tolerant to furfural and to hydroxymethylfurfural (HMF), xenobiotic agents regenerated during acidic pretreatment of biomass.

The fermentation of C5 and C6 is performed preferentially by a single Deinococcus strain, that is Deinococcus strain being able to co-ferment C5 and C6. In addition, Deinococcus strains have preferentially cellulolytic or xylanolytic activity and more preferentially both cellulolytic and xylanolytic activities efficient enough to reduce the amount of exogenous enzymes and sufficient enough to avoid adding exogenous depolymerizing enzymes.

Most preferred Deinococcus bacteria for use in the invention have the following characteristics:
Growth at a temperature comprised between 40-65° C., preferably 40-60° C., more preferably 40-55° C., and in particular at 48° C.;
cellulolytic or xylanolytic activity;
growth at a pH range of 3-9, preferably 4-9, more preferably 5-6, even more preferably at about 6; and
simultaneous utilization of C6 and C5 sugars (i.e. co-assimilation of C5 and C6 sugars).

In a most preferred embodiment, Deinococcus bacteria for use in the method have the following additional characteristics:
ethanol tolerance;
tolerance to furfural and/or hydroxymethylfurfural (HMF); and/or
ability to ferment xylobiose and xylotriose, and/or
ability to ferment cellobiose and cellotriose and/or
amylolytic activity.

Process Configuration

The present invention provides improved methods of transforming biomass using Deinococcus bacteria. As indicated, the invention preferably utilizes thermophile Deinococcus bacteria, under particular treatment conditions, and is able to effectively transform biomass under industrially-compatible conditions. The invention may be used to produce biofuels, isoprenoids or organic acids, as well as any valuable molecules from biomass. The invention is particularly advantageous since different embodiments can be implemented, depending on nature of the biomass and type of production that is needed. The method of the invention provides several advantages such as improved production yields, decreased quantity of use (and cost) of depolymerizing enzymes, decreased risk of microbial contaminations, fully integrated method, etc.

In a particular embodiment, the method is a three-step SHF (“Separate Hydrolysis and Fermentation”) process which comprises:

a) treating the biomass or providing a treated biomass,

b) hydrolyzing the treated biomass in the presence of at least one added source of exogenous cellulase and/or hemicellulase, and

c) fermenting the hydrolyzed biomass using one or more Deinococcus bacteria.

During the process, any product of interest may be collected.

According to such embodiment, pre-treated biomass is hydrolyzed to glucose and subsequently fermented, in a separate reactor unit. The hydrolysis may be performed with added cellulase(s). When lignocellulosic biomass is rich in pentose such as hardwood and crop residues, pentose rich fraction (hemicelluloses hydrolyzates) may be converted in ethanol. Fermentation of soluble pentose such as xylose may be performed subsequently to the fermentation of glucose in a second separate pentose-fermentation reactor or co-fermented with the hexose-reactor when the Deinococcus strain is able to co-ferment the glucose and xylose. The hemicellulose hydrolyzates may be also separated during pre-treatment and fermented in a separate pentose-fermentation reactor simultaneously to glucose-fermentation. Alternatively, or in addition, hemicellulase(s) may be added in the hydrolyzing reactor to generate pentoses which are fermented subsequently in the pentose-hexose fermentation reactor. Hemicellulases and cellulases may be added simultaneously or sequentially in the same hydrolyzing reactor. The major advantage is that the hydrolysis and fermentation step are performed in optimal conditions of temperature and pH. In that case, any Deinococcus (meso- or thermophile) may be used for the fermentation. The hydrolysis process can take from one to four days. The fermentation of C5 and C6 may be performed by the same Deinococcus strain which preferentially co-ferment glucose and xylose or alternatively by two separate strains or a combination of Deinococcus strains with any ethanol-producer micro-organisms, one having the ability to consume C5 and the other the C6.

In another particular embodiment, the method is a two-step SSF (“Simultaneous Saccharification and Fermentation”) process which comprises:

a) treating the biomass or providing a treated biomass, and

b) concomitantly hydrolyzing and fermenting the treated biomass in a same reactor using at least one Deinococcus bacterium in the presence of at least one added source of exogenous cellulase or hemicellulase.

During the process, any product of interest may be collected.

According to such embodiment, hydrolysis of pre-treated biomass and fermentation of the sugars are performed in the same reactor. The advantage of such an embodiment is that the glucose produced is directly consumed by the Deinococcus bacteria in the culture medium, minimizing the inhibitory effect of glucose and cellobiose on the depolymerizing enzymes by keeping a low concentration of these sugars in the medium. Indeed, at a cellobiose concentration as low as 6 g/l, the activity of cellulase is reduced by 60% (Taherzadeh M J and Karimi K. Bioresources 2007). Furthermore, glucose is a strong inhibitor of beta-glucosidase; at a level of 3 g/L of glucose, the activity is reduced by 75% (Philippidis and Smith 1995). Such an embodiment also yields higher amount of products from biomass and can require lower amounts of enzymes minimizing the risk of microbial contamination in the reactor. In addition, this is also lowered thanks to the presence of ethanol.

The invention shows that Deinococcus bacteria can be fully active and grown under temperature and pH conditions that are optimal for enzymatic hydrolysis and fermentation, thereby allowing optimal SSF. Using thermophile Deinococcus growing between 40-65° C., preferably between 40-55° C., SSF under optimal hydrolysis and optimal fermentation conditions can be performed. Furthermore, the invention shows that Deinococcus can tolerate high temperature and ethanol concentration, further decreasing the possibility of microbial contamination.

An object of the invention therefore resides in a SSF process comprising introducing treated biomass in a reactor, adding to the reactor a cellulase or a hemicellulase activity and a thermophile Deinococcus bacterium, maintaining the reactor at a temperature comprised between 40 and 65° C., preferably between 40 and 55° C., and collecting products from the reactor.

In case of pentose rich-hydrolyzates, the soluble pentose rich fraction (hemicelluloses hydrolyzate) can be converted in ethanol in a separate pentose-fermenting reactor prior to or after hexose-fermentation. Alternatively, the soluble pentose fraction may be separated after pre-treatment of biomass and fermented simultaneously with the hexose fermentation in a separate pentose-fermentation reactor. The fermentation of C5 and C6 may be performed by the same Deinococcus strain or by two distinct strains one having the ability to consume C5 and the other the C6. In addition, Deinococcus strains have preferentially cellulolytic activity, enough efficient to reduce the amount (and so on, the cost) of cellulases used in the process.

In a preferred variant, the SSF process is a SSCF (Simultaneous Saccharification and CO-Fermentation”) process wherein both pentose and hexose are co-fermented in a single reactor. Using Deinococcus bacteria, the inventors show that the hydrolyzed hemicelluloses during pre-treatment and solid cellulose do not need to be separated after pre-treatment, allowing the hemicellulose sugars (mainly soluble pentose) to be converted together with the cellulose in the same reactor. In that case, only addition of cellulase is required to hydrolyze the cellulose. Alternatively, depending on the choice of pre-treatment, the hemicellulose may be intact (or partially intact) after pre-treatment. Therefore, the entire slurry of pretreated biomass (hemicelluloses and cellulose) may be hydrolyzed with a mixture of enzyme comprising cellulases and hemicellulases or hydrolyzed with the individual enzymes taken separately and added simultaneously in the same hydrolyzing-fermention reactor. The fermentation of C5 and C6 is performed preferentially by a single Deinococcus strain able to co-ferment C5 and C6 (or alternatively by two separate strains or a combination of Deinococcus strains, one having the ability to consume C5 and the other the C6). In addition, Deinococcus have preferentially cellulolytic activity and more preferentially both cellulolytic and xylanolytic activities efficient enough to reduce the amount (and so on the cost) of hydrolyzing enzyme used in the process.

An object of the invention therefore resides in a SSCF process comprising introducing treated biomass in a reactor, adding to the reactor a cellulase and a hemicellulase activity and a thermophile Deinococcus bacterium, maintaining the reactor at a temperature comprised between 40 and 65° C., preferably between 40 and 55° C., and collecting products from the reactor.

In another embodiment, the process is a two-step CBP (“Consolidated Bioprocessing”) or DMC (“Direct Microbial Conversion”) process comprising:

a) treating the biomass or providing a treated biomass, and

b) concomitantly hydrolyzing and fermenting the treated biomass, in a same reactor, using at least one Deinococcus bacterium which produces a cellulase and/or hemicellulase activity.

During the process, any product of interest may be collected. Also, additional enzymes or sources of enzymes may be added to the reactor to further improve the process, if needed.

According to a preferred embodiment, all enzymes or reagents needed in the process are produced by Deinococcus bacteria in the reactor. The invention shows that Deinococcus bacteria are able not only to cause effective fermentation but also to produce (or be engineered to produce) suitable amounts of depolymerizing enzymes (such as cellulase(s) and/or hemicellulase(s)) under suitable industrial conditions, compatible with effective biomass transformation.

In this regard, a more specific object of the invention therefore resides in a CBP process comprising introducing a treated biomass in a reactor, adding to the reactor a thermophile Deinococcus bacterium, said bacterium having cellulase and/or hemicellulase activity, maintaining the reactor at a temperature comprised between 40 and 65° C., preferably between 40 and 55° C., and collecting products from the reactor.

The reactions can be performed under fedbatch or batch or continuous culture condition systems, preferentially fedbatch.

As indicated above, the invention may be used with any biomass, particularly lignocellulosic biomass. It is suited to transform biomass for producing molecules of interest such as peptides, polypeptides, proteins, nucleic acids, small molecules, including alcohols, organic acids, metabolites, polymers, etc.), lipids, vegetal extracts, and the like.

In a preferred embodiment, the invention is directed to the production of an alcohol from lignocellulosic biomass using a Deinococcus bacterium in a SSF, SSCF or CBP process.

In another preferred embodiment, the invention is directed to the production of an isoprenoid compound from lignocellulosic biomass using a Deinococcus bacterium in a SSF, SSCF or CBP process.

A particular embodiment of the invention comprises:

a) treating a lignocellulosic biomass or providing a treated lignocellulosic biomass,

b) concomitantly hydrolyzing and fermenting the treated biomass in a same reactor by exposing the biomass to at least a Deinococcus bacterium in the presence of at least one added source of exogenous cellulase and/or hemicellulase, at a reaction temperature comprised between 40 and 65° C., preferably between 40 and 55° C., and

c) collecting an alcohol produced during the process, preferably ethanol.

Another particular embodiment of the invention comprises:

a) treating a lignocellulosic biomass or providing a treated lignocellulosic biomass,

b) concomitantly hydrolyzing and fermenting the treated biomass in a same reactor by exposing the biomass to at least a Deinococcus bacterium at a reaction temperature comprised between 40 and 65° C., preferably between 40 and 55° C., said Deinococcus bacterium producing a cellulase and/or hemicellulase, and

c) collecting an alcohol produced during the process, preferably ethanol.

In a preferred embodiment, the treated biomass is an acid treated biomass, more preferably a biomass treated under mild acid condition and, optionally, with steam explosion or hot water.

Another particular embodiment of the invention comprises:

a) heating a starch-containing biomass, or providing a heated starch-containing biomass,

b) concomitantly hydrolyzing and fermenting the treated biomass in a same reactor by exposing the biomass to at least a Deinococcus bacterium in the presence of at least one added source of exogenous amylase, at a reaction temperature comprised between 40 and 65° C., preferably between 40 and 55° C., and

c) collecting a product produced during the process, preferably ethanol or an isoprenoid compound.

Another particular embodiment of the invention comprises:

a) heating a starch-containing biomass, or providing a heated starch-containing biomass,

b) concomitantly hydrolyzing and fermenting the treated biomass in a same reactor by exposing the biomass to at least a Deinococcus bacterium at a reaction temperature comprised between 40 and 65° C., preferably between 40 and 55° C., said Deinococcus bacterium producing an amylase, and

c) collecting a product produced during the process, preferably ethanol or an isoprenoid compound.

In a particular embodiment, the starch-containing biomass is a starch-rich biomass such as domestic biowastes. The pretreatment may comprise a heat & pressure treatment such as a jet cooker.

Further aspects of the invention are disclosed in the following experimental section, which is illustrative.

### EXAMPLES

I—Transformation of Wheat Straw by SHF Process Using Deinococcus Bacteria.

a) Dilute Acid Pretreatment

40 g of dried wheat straw are put in contact with 500 mL of 2% (w/v) sulfuric acid (H₂SO₄). The acid impregnated biomass is then incubated 15 min at 30° C. under shaking (150 rpm) and autoclaved at 121° C. during 45 min. The pH of the mixture is then adjusted with a solution of NaOH (20 M) to neutralize the acid. The acid treatment increases the cellulose power of the biomass.

b) Enzymatic Hydrolysis

The pre-treated biomass of Ia) above is contacted with a mix of enzymes mixture comprising 203.3 mg of cellulase (30 FPU/biomass dried matter), 192 mg of beta-glucosidase (1.5 U/biomass dried matter), and 0.1 g of Viscozyme (10 FPU/biomass dried matter). The enzymes used are as follows:
Cellulase (EC 3.2.1.1) from Trichoderma reesei—(SIGMA C8546≧6 Units/mg)
Beta-glucosidase (EC 3.2.1.21) from almonds—(SIGMA ref.49290-1G—8.1 U/g)
Viscozyme—(SIGMA ref. v2010—100 FBG/g, 7% w/w, 1.21 g/ml) which is a mixture comprising a Beta-glucanase (endo-1,3(4), a cellulase and a xylanase.

The hydrolysis is performed at 45° C. under shaking 150 rpm during 72 h.

c) Fermentation

Deinococcus are cultivated either in batch, fedbatch or continuous mode at different growth rate, between 0.02 to 0.8 h⁻¹. Deinococcus are added to the pretreated and hybrolyzed biomass slurry of Ib) in a reactor. The temperature and pH are kept constant at fixed temperature between 40° C. and 60° C., and at fixed pH between 4 and 9 respectively. Agitation is defined in order to ensure a good transport in the bioreactor.

We can distinguish two kinds of processes: first one is an anaerobic process that implies the production of molecules that is associated to ATP production such as ethanol or lactate (“energy producing product”), and the second one is an aerobic process that implies the production of molecules that is associated to ATP consumption such as carotenoids (“Energy consuming product”).

Energy Producing Product Conditions:

Aeration of N2/O2 is used in order to satisfy minimal oxygen requirement in microaerobic process and to ensure stripping of volatile compound thanks to aeration between 0.1 to 2 vvm.

Aeration of N2 is used in anaerobic process in order to ensure stripping of volatile compound thanks to aeration between 0.1 to 2 vvm.

Energy Consuming Product Conditions

Aeration of air or air/O2 mix or N2/O2 mix is used in order to satisfy minimal oxygen requirement in aerobic process and to ensure stripping of volatile compound thanks to aeration between 0.1 to 2 vvm.

The process allows effective transformation of biomass to generate valuable products.

II—Transformation of Bio-Wastes by SHF Process Using Deinococcus Bacteria.

a) Dilute Acid Pretreatment

40 g of dried bio wastes are put in contact with 500 mL of 2% (w/v) sulfuric acid (H₂SO₄). The acid impregnated biomass is then incubated 15 min at 30° C. under shaking (150 rpm) and autoclaved at 121° C. during 45 min. The pH of the mixture is then adjusted with a solution of NaOH (20 M) to neutralize the acid. The acid treatment increases the cellulose power of the biomass.

b) Enzymatic Hydrolysis (Saccharification)

The pre-treated biomass of IIa) above is contacted with a mix of enzymes mixture comprising 203.3 mg of cellulase (30 CFU/biomass dried matter), 192 mg of beta-glucosidase (1.5 U/biomass dried matter), and 0.1 g of Viscozyme (10 CFU/biomass dried matter). The enzymes used are as described in example Ib). The hydrolysis is performed at 45° C. under shaking 150 rpm during 72 h.

c) Fermentation

Deinococcus are cultivated either in batch, fedbatch or continuous mode at different growth rate, between 0.02 to 0.8 h⁻¹. Thermophile Deinococcus are added to the pretreated and hybrolyzed biomass slurry of IIb) in a reactor. The temperature and pH are kept constant at fixed temperature between 40° C. and 60° C., and at fixed pH between 4 and 9 respectively. Agitation is defined in order to ensure a good transport in the bioreactor.

Aeration of N2/O2 is used in order to satisfy minimal oxygen requirement in microaerobic process and to ensure stripping of volatile compound thanks to aeration between 0.1 to 2 vvm. Alternatively, aeration of N2 is used in anaerobic process in order to ensure stripping of volatile compound thanks to aeration between 0.1 to 2 vvm.

The process allows effective transformation of biomass to generate valuable products.

III—Transformation of Corn Stover by SSCF Process Using Deinococcus Bacteria.

a) AFEX Treatment

Corn stover is milled to a particle size of 4 mm and then pre-treated using AFEX, under the following conditions:
Ammonia to Biomass loading: 1 g/g dry biomass
Water Loading: 0.6 g/g dry biomass
Temperature: 140° C.
Residence time: 15 min

AFEX pre-treated corn stover is then used for saccharification and fermentation without any washing or detoxification.

b) Simultaneous Saccharification and Co-fermentation

Simultaneous saccharification and co-fermentation are conducted by incubating the pre-treated corn stover biomass with a mix of enzymes and a thermophile Deinococcus strain able to co-ferment glucose and xylose, in 500 mL reactors with a cultivation volume of 50 mL. The initial OD600 for Deinococcus is 0.4.

The reactors are incubated at 48° C. and the stirring speed is 250 rpm. The pH is adjusted to 6 with MES Sodium. AFEX pre-treated substrate loading is 0.5% (w/w). The enzyme mix comprises Spezyme CP (Genencor Inc., Rochester, N.Y.) 22.4 mg protein/g glucan (15 FPU/g glucan), Novozyme 188 (Sigma-Aldrich, St. Louis, Mo.) 38.4 mg protein/g glucan (64 pNPGU/g glucan), Multifect xylanase 2.6 mg protein/g glucan, and Multifect pectinase (Genencor Inc) 4.7 mg protein/g glucan. The total incubation time is 3-4 days.

Ethanol yield above 0.4 g ethanol/g glucose+xylose can be obtained.

IV—Transformation of Wheat Straw by CBP Process Using Deinococcus Bacteria.

Dilute acid pre-treatment of wheat straw was performed as follows: 40 g of dried matter is put in contact with 500 mL of 2% (w/v) sulfuric acid (H₂SO₄). The acid impregnated biomass is then incubated 15 min at 30° C. under shaking (150 rpm) and autoclaved at 121° C. during 45 min. The pH of the mixture is then adjusted with a solution of NaOH (20 M) to neutralize the acid.

The bioprocessing conditions are as follows: temperature 48° C., pH 6.00, and stirring 250 rpm. Reactions are conducted in 500 mL reactors with a 50 mL working volume. Reactors are loaded with 0.5 g of biomass on a dry basis and 47.25 mL of culture media. The bioreactor for CBP process containing the pre-treated wheat straw is inoculated with a single cellulolytic and xylanolytic Deinococcus strain with an initial OD₆₀₀ of 0.4. The batch mode of CBP operation is performed for 3-4 days.

1-23. (canceled)

24. A method of transforming a biomass, comprising
a) subjecting the biomass to a treatment to increase the hydrolytic power thereof;
b) simultaneously with or subsequently to step a), exposing the biomass to an enzymatic activity that degrades polysaccharides in the biomass; and
c) simultaneously with or subsequently to step b), exposing the biomass to a Deinococcus bacterium or an extract thereof.

25. The method of claim 24, wherein step a), b) and c) are conducted simultaneously.

26. The method of claim 24, wherein step b) and c) are performed simultaneously, after step a).

27. The method of claim 24, wherein step a) comprises subjecting the biomass to steam, heat, pressure, pressurized hot water, supercritical water, mild acid, strong acid, mild or strong alkaline treatment, or ammonia fiber explosion, or a combination thereof.

28. The method of claim 27, wherein step a) comprises an acid treatment at a pH comprised between 3 and 6.

29. The method of claim 27, wherein step a) (further) comprises a transient thermic and/or pressure treatment.

30. The method of claim 24, wherein the biomass is pretreated to remove fine particles.

31. The method claim 24, wherein the treated biomass is washed after treatment step a).

32. The method of claim 24, wherein exposing the biomass to an enzymatic activity comprises adding one or several enzymes to the biomass, and/or inducing or allowing expression of one or several enzymes from a microorganism.

33. The method of claim 32, wherein said microorganism is a Deinococcus bacterium of step c).

34. The method of claim 24, wherein step c) comprises exposing the biomass to a thermophilic Deinococcus bacterium.

35. The method of claim 24, wherein the Deinococcus bacterium comprises a recombinant nucleic acid encoding one or several enzymes that degrade polysaccharides in the biomass.

36. The method of claim 24, wherein steps a) to c) are performed in a liquid medium, and may optionally further include a solid/liquid separation.

37. The method of claim 24, wherein the pH in steps a) to c) is between 3 and 9.

38. The method of claim 24, wherein steps b) and c) are performed at a temperature between 40 and 65° C.

39. The method of claim 24, which comprises providing a biomass and exposing the biomass to a Deinococcus bacterium in an acid medium in the presence of one or several enzymes that increase cellulosic power.

40. The method of claim 39, wherein the enzymes are produced by the Deinococcus bacterium.

41. The method of claim 24, wherein the biomass comprises cellulose, hemicellulose, lignin, xylan, starch or pectin.

42. The method of claim 41, wherein the biomass is a corn or wheat biomass.

43. The method of claim 41, wherein the biomass is corn stover, corn cobs, wheat straw, bagasse, softwood, hardwood, citrus peels, domestic biowaste, urban biowaste, or municipal solid wastes or a fraction thereof.

44. A method of producing an alcohol, comprising:
a) subjecting a biomass to a treatment to increase the hydrolytic power thereof, or providing a treated biomass having hydrolytic power;
b) simultaneously with or subsequently to step a), exposing the biomass to an enzymatic activity that degrades polysaccharides in the biomass;
c) simultaneously with or subsequently to step b), exposing the biomass to a Deinococcus bacterium or an extract thereof, and
d) collecting the alcohol produced.

45. The method of claim 44, wherein the alcohol is ethanol, propanol or butanol.

46. A method for producing a product of interest from a lignocellulosic biomass, comprising:
a) treating the biomass to increase the hydrolytic power or providing a treated biomass having hydrolytic power,
b) concomitantly hydrolyzing and fermenting the treated biomass in a same reactor by exposing said treated biomass to at least one Deinococcus bacterium in the presence of at least one added source of exogenous cellulase and/or hemicellulase and/or amylase, and
c) collecting a product of interest produced.

47. The method of claim 46, wherein the product of interest is a biofuel, an isoprenoid, a carotenoid, an organic acid, a protein or a vegetal extract.

48. A method for producing a product of interest from a lignocellulosic biomass, comprising:
a) treating the biomass to increase the hydrolytic power or providing a treated biomass having hydrolytic power,
b) concomitantly hydrolyzing and fermenting the treated biomass in a same reactor by exposing said treated biomass to at least one Deinococcus bacterium, said bacterium having cellulase and/or hemicellulase and/or amylase activity, and
c) collecting a product of interest produced.

49. The method of claim 48, wherein the product of interest is a biofuel, an isoprenoid, a carotenoid, an organic acid, a protein or a vegetal extract.
